(19) United States
(12) Patent Application Publication
(10) Pub. No.: US 2025/0332446 A1
(43) Pub. Date: Oct. 30, 2025

(54) TRANSLATING CLINICAL INPUTS INTO RADIOTHERAPY TREATMENT PLANNING DATA

(71) Applicants: Elekta (Shanghai) Technology Co., Ltd., Shanghai (CN); Elekta, Inc., Atlanta, GA (US)

(72) Inventors: Xiaotian Huang, Shanghai (CN); Laurent Collignon, Nivelles (BE)

(21) Appl. No.: 19/073,549

(22) Filed: Mar. 7, 2025

(30) Foreign Application Priority Data
Apr. 29, 2024 (CN) 202410536101.4

(51) Int. Cl. A61N 5/10; G16H 10/60; G16H 20/40

(52) U.S. Cl. CPC A61N 5/1031; G16H 10/60; G16H 20/40; A61N 2005/1074

(57) ABSTRACT

A system and method for generating automation commands for a treatment planning system used with radiotherapy treatment, includes: receiving text input from a user, the text input including information relating to a radiotherapy treatment plan of a patient; determining, based on parsing the text input, commands of the treatment planning system and command parameters to be used by the treatment planning system; generating scripting with a generative chatbot based on the text input, wherein the scripting includes the commands and the command parameters; and outputting the scripting for automation of the treatment planning system in connection with establishing the radiotherapy treatment plan for the radiotherapy treatment of the patient.

### PRIORITY CLAIM

This application claims the benefit of priority to China Patent Application No. 202410536101.4, filed Apr. 29, 2024, and titled “Methods and Systems for Generating Automation Commands, and Computer-Readable Medium”, which is incorporated herein by reference in its entirety.

### TECHNICAL FIELD

Embodiments herein relate to methods and systems for processing medical data. In particular, the methods and systems are directed to processing medical data associated with radiotherapy.

### BACKGROUND

Radiotherapy or radiation therapy can be described as the use of ionizing radiation to damage or destroy unhealthy cells in both humans and animals. Unhealthy cells may include cancerous cells, for example. The ionizing radiation may be directed to tumors on the surface of the skin or deep inside the body. Common forms of ionizing radiation include X-rays and charged particles. An example of a radiotherapy technique is Gamma Knife® or Leksell Gamma Knife® where a patient is irradiated using a number of lower-intensity gamma rays that converge with higher intensity and high precision at a targeted region (e.g., a tumor). Another example of radiotherapy comprises using a linear accelerator (“linac”), whereby a targeted region is irradiated by high-energy particles (e.g., electrons, high-energy photons, and the like). In another example, radiotherapy is provided using a heavy charged particle accelerator (e.g., protons, carbon ions, and the like).

Computer systems are used to create treatment plans that control and personalize the output of radiotherapy to a particular patient. Treatment plan data may include details of the treatment delivery, such as the number of beams, the machine, the modality, the beam energy, and the machine output in monitor units (MU), among other aspects. The software applications that create and modify treatment plans are referred to as “treatment planning systems”, and these systems often include sophisticated functionality to calculate, customize, verify, and optimize the details of a specific radiotherapy treatment and the treatment plan data.

Treatment plans may be created by a treatment planning system based on data including but not limited to: patient data (e.g., an electronic medical record (EMR) or electronic health record (EHR)), medical imaging data, treatment plan information, dose-volume histograms (DVHs), dose information, dosimetric metrics, clinical outcomes, instructions for use (IFUs), and the like. Medical imaging data, for example, may include information relating to certain anatomical structures and the treatment to be delivered to the anatomical structures (e.g., planned target volume, target, organ(s) at risk, etc.).

Translating clinical needs from planned protocols into objectives and controls for a specific treatment plan of a patient is not straightforward. With conventional medical processes, this involves discussions between the radiation oncologist, medical physicist, and planners to assess the feasibility and find the right objectives and constraints to input into the treatment planning system. Accordingly, there is a need for improved methods and systems for analyzing medical data and inputs associated with treatment planning, to enable users and computing systems to correctly process medical data, to improve the accuracy of treatment, and to reduce the number of computation operations in implementing systems.

### BRIEF DESCRIPTION OF DRAWINGS

Systems and methods in accordance with non-limiting examples will now be described with reference to the accompanying figures in which:

FIG. 1 depicts a radiotherapy system adapted to perform radiotherapy plan processing operations using the approaches discussed herein.

FIG. 2 depicts a use case for invoking a treatment planning automation workflow.

FIG. 3 depicts a sequence of operations for treatment planning automation, based on treatment planning system scripting produced from natural language.

FIG. 4 depicts a detailed processing workflow of treatment planning automation workflow, for conversion of human commands into executable commands.

FIG. 5 depicts a schematic illustration of an example language model usable by a chatbot.

FIG. 6 depicts a flowchart of an example method for generating automation commands for a treatment planning system, based on automation provided from the approaches discussed herein.

FIG. 7 depicts a flowchart of an example method for providing a radiation therapy treatment, based on automation provided from the approaches discussed herein.

FIG. 8 is a block diagram of an apparatus, device, system, or machine according to various embodiments.

### DETAILED DESCRIPTION

The following describes methods and systems that improve the generation and operation of radiotherapy treatment planning, through the processing of natural language to generate executable scripts or other data output that controls a radiotherapy treatment planning system. This can help limit human interaction with radiotherapy treatment planning software, while accurately translating clinical needs into inputs for the treatment planning application to produce improved output for a radiotherapy machine. In addition to clear technical benefits, the automated generation of executable scripts or software control instructions, from natural language, also provides a beneficial process that can help clinical users save time while improving accuracy and efficiency.

The following methods and systems can be invoked by a variety of users, including dosimetrists or therapy planning personnel. Such personnel can invoke automation and improved computer functions in a treatment planning system without knowing how to write source code or how to implement complex medical requirements specified by radiation oncologists. As a result, the methods and systems can be used to automatically control and operate a treatment planning system, producing treatment plans and data that control a radiation therapy process and a planning workflow with improved outcomes for a patient.

In particular, the methods and systems include the generation of scripts based on the entry of a user command (also referred to as a query, request, or prompt), provided in natural language using a chatbot. A chatbot is a type of software agent configured to converse with a user via text or voice. A chatbot may also be referred to as a conversational agent or system, a smart assistant, or an artificial intelligence (AI) agent. Natural language refers to language that occurs naturally in human communication, provided in spoken or written form. In this context, natural language might occur in the context of a user command that is entered to ask the treatment planning system, in simple words, to perform some complex type of action (or series of actions). Natural language is different from a structured language such as a computer programming language. Due to the nuance of human language and complexity of programming languages, the conversion of natural language commands into useful computer programming commands—in a radiotherapy planning setting—has not been addressed by existing approaches.

The following methods and systems thus introduce text/voice chatbot functions that can be used to produce scripting (e.g., scripts with computer programming commands) to achieve automation in a treatment planning system. Additionally, these text/voice chatbot functions can be used to translate clinical needs into automation commands and other inputs for the treatment planning system, as part of individual commands, a conversation, or a context or session associated with a patient or treatment use case. As an example, users can provide natural language by writing or speaking to an AI agent, in an ongoing conversation about what actions need to be undertaken for a patient for treatment planning. Based on these inputs, the AI agent can produce scripting and commands, to achieve the tasks automatically (or, with simpler forms of human interaction) in the treatment planning software.

With the present methods and systems, any user (e.g., a treatment planner, dosimetrist, clinician, health care worker, or analyst) could invoke automation from natural language to produce smart automations and adaptation of treatment planning operations. A dosimetrist or planner could use the automation without knowing how to write code or perform complex treatment planning, reaching the same outcome and sophistication as skilled radiation oncologists without training or expertise. In the same way that a user would converse with a human colleague using natural language, the natural language inputs can be interpreted by the system and used to control a variety of subsystems with accurate scripting commands.

By using natural language (instead of, e.g., a computer programming language) the ease of use is increased, and more efficient computing operations can be achieved. The methods and systems herein thus address a technical problem arising in the field of processing medical data (in particular, radiotherapy medical data), namely, how to facilitate the accurate processing of medical data, based on received and interpreted commands from a user in natural language, while providing a precise control of systems via program instructions. This benefit can be leveraged especially when a user presents a complex wishlist or sequence of commands, or attempts to invoke unique or customized actions in treatment planning software.

Further explanation of natural language processing is provided after an overview of the present systems and methods, including an overview of radiotherapy treatment and treatment planning systems.

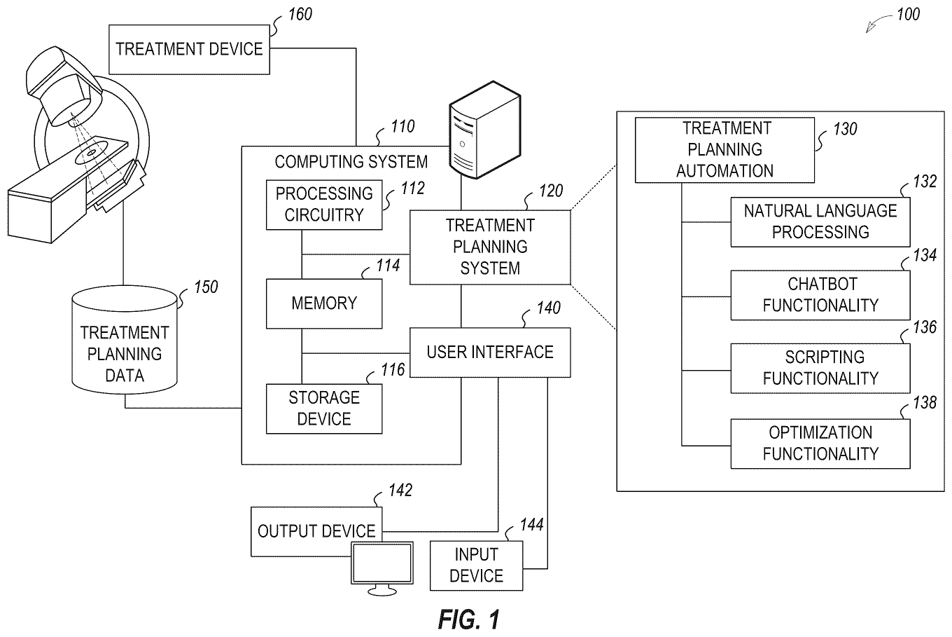

FIG. 1 illustrates a radiotherapy system 100 adapted to perform radiotherapy plan processing operations using one or more of the approaches discussed herein. These radiotherapy plan processing operations are performed to enable the radiotherapy system 100 to provide radiation therapy to a patient based on specific aspects of captured medical imaging data and therapy dose calculations or radiotherapy machine configuration parameters. Specifically, the following processing operations may be implemented as part of a treatment planning system 120 (also known as a “TPS”) for developing a radiotherapy treatment plan based on natural language commands. It will be understood, however, that many variations and use cases of the treatment planning system 120 and automation operations may be provided, such as in response to data optimization, visualization, and other medical evaluative and diagnostic operations.

The radiotherapy system 100 includes a radiotherapy data processing computing system 110 that hosts a treatment planning system 120. The radiotherapy data processing computing system 110 may be connected to a network (not shown), and such network may be connected to the Internet. For instance, a network can connect the radiotherapy data processing computing system 110 with one or more private and/or public medical information sources (e.g., a radiology information system (RIS), a medical record system (e.g., an electronic medical record (EMR)/electronic health record (EHR) system), an oncology information system (OIS)), one or more image data sources, an image acquisition device (e.g., an imaging modality). In the depicted example, the radiotherapy data processing computing system 110 is operably coupled to a treatment planning data source 150 (e.g., a database that stores treatment plans) and a treatment device 160 (e.g., a radiation therapy device that implements the treatment plans).

As an example, the radiotherapy data processing computing system 110 can be configured to receive a treatment goal of a subject (e.g., anatomical areas to deliver treatment) and generate a radiotherapy treatment plan by executing instructions or data within a treatment planning system 120, as part of operations to generate treatment plans to be used by the treatment device 160 and/or output on the output device 142. In an embodiment, the treatment planning system 120 is a software application or software platform that includes programmed functionality to generate, validate, and optimize a radiotherapy treatment plan for each patient.

The radiotherapy data processing computing system 110 may include processing circuitry 112, memory 114, a storage device 116, and other hardware and software-operable features such as a user interface 140, a communication interface (not shown), and the like. The storage device 116 may store transitory or non-transitory computer-executable instructions, such as an operating system, radiation therapy treatment plans, training data, software programs (e.g., image processing software, image or anatomical visualization software, artificial intelligence (AI) or ML implementations and algorithms such as provided by deep learning models, ML models, and neural networks (NNs), etc.), and any other computer-executable instructions to be executed by the processing circuitry 112.

In an example, the processing circuitry 112 may include at least one processing device, such as one or more general-purpose processing devices such as a microprocessor, a central processing unit (CPU), a graphics processing unit (GPU), an accelerated processing unit (APU), or the like. More particularly, the processing circuitry 112 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processing circuitry 112 may also be implemented by one or more special-purpose processing devices such as an application-specific integrated circuit (ASIC), a field programmable gate array (FPGA), a digital signal processor (DSP), a System on a Chip (SoC), or the like.

As would be appreciated by those skilled in the art, in some examples, the processing circuitry 112 may be a special-purpose processor rather than a general-purpose processor. The processing circuitry 112 may include one or more known processing devices, such as a microprocessor from the Pentium™, Core™, Xeon™ or Itanium™ family manufactured by Intel®, the Turion™, Athlon™, Sempron™ Opteron™, FX™, Phenom™ family manufactured by AMD™, or any of various processors manufactured by Sun Microsystems. The processing circuitry 112 may also include graphical processing units such as a GPU device provided from the GeForce®, Quadro®, Tesla® family manufactured by Nvidia™, GMA, Arc™ family manufactured by Intel®, or the Radeon™ family manufactured by AMD®. The processing circuitry 112 may also include accelerated processing units such as those incorporated into the Xeon™ family manufactured by Intel®.

In some examples, the processing circuitry 112 may include or be arranged into a parallel processing configuration. For instance, a set of graphical processing units (e.g., GPU cores, units, devices, or cards from the GeForce®, Quadro®, Tesla® family manufactured by Nvidia®, GMA, Arc™ family manufactured by Intel®, or the Radeon™ family manufactured by AMD®) may be arranged to perform highly parallel or repetitive computing tasks simultaneously. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one physical (circuitry-based) or software-based processor (for example, a multi-core design or a plurality of processors each having a multi-core design). The processing circuitry 112 can execute sequences of transitory or non-transitory computer program instructions, stored in memory 114, and accessed from the storage device 116, to perform various operations, processes, and methods that will be explained in greater detail below. It should be understood that any component in the radiotherapy system 100 may be implemented separately and operate as an independent device and may be coupled to any other component in the radiotherapy system 100 to perform the techniques described in this disclosure.

The memory 114 may comprise read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a compact disc read-only memory (CD-ROM), a digital versatile disc (DVD) or other optical storage, a cassette tape, other magnetic storage device, or any other non-transitory medium that may be used to store information including images, training data, one or more ML model(s) or technique(s) parameters, data, or transitory or non-transitory computer executable instructions (e.g., stored in any format) capable of being accessed by the processing circuitry 112, or any other type of computer device. For instance, the computer program instructions can be accessed by the processing circuitry 112, read from the ROM, or any other suitable memory location, and loaded into the RAM for execution by the processing circuitry 112.

The storage device 116 may constitute a drive unit that includes a transitory or non-transitory machine-readable medium on which is stored one or more sets of transitory or non-transitory instructions and data structures (e.g., software) embodying or utilized by any one or more of the methodologies or functions described herein (including, in various examples, the treatment planning system 120 and the user interface 140). The instructions may also reside, completely or at least partially, within the memory 114 and/or within the processing circuitry 112 during execution thereof by the radiotherapy data processing computing system 110, with the memory 114 and the processing circuitry 112 also constituting transitory or non-transitory machine-readable media.

The memory 114 and the storage device 116 may constitute a non-transitory computer-readable medium. For example, the memory 114 and the storage device 116 may store or load transitory or non-transitory instructions for one or more software applications on the computer-readable medium. Software applications stored or loaded with the memory 114 and the storage device 116 may include, for example, an operating system for common computer systems as well as for software-controlled devices. The radiotherapy data processing computing system 110 may also operate a variety of software programs comprising software code for implementing the treatment planning system 120, the treatment planning automation workflow 130, and the user interface 140. Further, the memory 114 and the storage device 116 may store or load an entire software application, part of a software application, or code or data that is associated with a software application, which is executable by the processing circuitry 112.

As non-limiting examples, the memory 114 and the storage device 116 may store, load, and manipulate one or more radiation therapy treatment plans, scripting commands, imaging data, segmentation data, treatment visualizations, histograms or measurements, one or more AI model data (e.g., weights and parameters of one or more ML model(s)), training data, labels and mapping data, and the like. It is contemplated that software programs may be stored not only on the storage device 116 and the memory 114 but also on a removable computer medium, such as a hard drive, a computer disk, a CD-ROM, a DVD, a Blu-Ray DVD, USB flash drive, an SD card, a memory stick, or any other suitable medium; such software programs may also be communicated or received over a network.

Although not depicted, the radiotherapy data processing computing system 110 may include a communication interface, network interface card, and communications circuitry. An example communication interface may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as an IEEE 802.11/Wi-Fi adapter), a telecommunication adapter (e.g., to communicate with 3G, 4G/LTE, and 5G networks and the like), and the like. Such a communication interface may include one or more digital and/or analog communication devices that permit a machine to communicate with other machines and devices, such as remotely located components, via a network. The network may provide the functionality of a local area network (LAN), a wireless network, a cloud computing environment (e.g., software as a service, platform as a service, infrastructure as a service, etc.), a client-server, a wide area network (WAN), and the like. For example, the network may be a LAN or a WAN that may include other systems (including additional image processing computing systems or image-based components associated with medical imaging or radiotherapy operations).

The processing circuitry 112 may be communicatively coupled to the memory 114 and the storage device 116, as the processing circuitry 112 is configured to execute computer-executable instructions stored thereon from either the memory 114 or the storage device 116. Particularly, treatment planning system 120 is adapted by the treatment planning automation workflow 130 to implement operations, programs, parameters, and other workflow actions for radiotherapy treatment, via scripting and computer software commands. The treatment planning system 120 can be controlled with the results of the treatment planning automation workflow 130 to produce new or updated treatment plan parameters (for deployment to the treatment planning data source 150 and/or presentation on the output device 142). The processing circuitry 112 may subsequently transmit the new or updated treatment plan parameters via a communication interface and the network to the treatment device 160, where the radiation therapy plan will be used to treat a patient with radiation via the treatment device 160, consistent with results of the treatment planning system 120 and the treatment planning automation workflow 130 (e.g., according to the processes discussed below).

The radiotherapy data processing computing system 110 may communicate with an external database through a network to send/receive a plurality of various types of data related to image processing and radiotherapy operations. For example, an external database may include machine data (including device constraints) that provides information associated with the treatment device 160, the image acquisition device, or other machines relevant to radiotherapy or medical procedures. Machine data information (e.g., control points) may include radiation beam size, arc placement, beam on and off time duration, machine parameters, segments, multi-leaf collimator (MLC) configuration, gantry speed, MRI pulse sequence, and the like. The external database may be a storage device and may be equipped with appropriate database administration software programs. Further, such databases or data sources may include a plurality of devices or systems located either in a central or a distributed manner.

The radiotherapy data processing computing system 110 can collect and obtain data, and communicate with other systems, via a network using one or more communication interfaces, which are communicatively coupled to the processing circuitry 112 and the memory 114. For instance, a communication interface may provide communication connections between the radiotherapy data processing computing system 110 and radiotherapy system components (e.g., permitting the exchange of data with external devices). For instance, the communication interface may, in some examples, have appropriate interfacing circuitry from an output device 142 or an input device 144 to connect to the user interface 140, which may be a hardware keyboard, a keypad, or a touch screen through which a user may input information into the radiotherapy system.

As an example, the output device 142 may include a display device that outputs a representation of the user interface 140 and one or more aspects, visualizations, or representations of the medical images, the treatment plans, and statuses of training, generation, verification, or implementation of such plans. The output device 142 may include one or more display screens that display medical images, interface information, treatment planning parameters (e.g., contours, dosages, beam angles, labels, maps, etc.), treatment plans, a target, localizing a target and/or tracking a target, or any related information to the user. The input device 144 connected to the user interface 140 may be a keyboard, a keypad, a touch screen, or any type of device that a user may use to the radiotherapy system 100. Alternatively, the output device 142, the input device 144, and features of the user interface 140 may be integrated into a single device such as a smartphone or tablet computer (e.g., Apple iPad®, Lenovo Thinkpad®, Samsung Galaxy®, etc.).

Furthermore, any and all components of the radiotherapy system may be implemented as a virtual machine (e.g., via VMWare, Hyper-V, and the like virtualization platforms) or independent devices. For instance, a virtual machine can be software that functions as hardware. Therefore, a virtual machine can include at least one or more virtual processors, one or more virtual memories, and one or more virtual communication interfaces that together function as hardware. For example, the radiotherapy data processing computing system 110, the image data sources, or like components, may be implemented as a virtual machine or within a cloud-based virtualization environment.

The treatment planning system 120 in the radiotherapy data processing computing system 110 implements the treatment planning automation workflow 130 consistent with the following examples. The treatment planning automation workflow 130 may implement operations for identifying and developing radiotherapy plans, based on capturing natural language inputs, and converting the natural language inputs into relevant operational computerized commands in the treatment planning system 120. In specific examples, treatment planning automation workflow 130 includes natural language processing 132 to convert and segment individual commands from language voice and text inputs; chatbot functionality 134 to interact with a user to identify relevant user commands, and to provide output with a user in a human conversational format to elicit the user commands; scripting functionality 136 to convert individual commands into a scripting program language usable with the treatment planning system 120 or another automation tool associated with the treatment planning system 120; and optimization functionality 138 to provide improvements for treatment plans, including to invoke built-in treatment planning optimizations and procedures or to implement custom automations associated with optimizations.

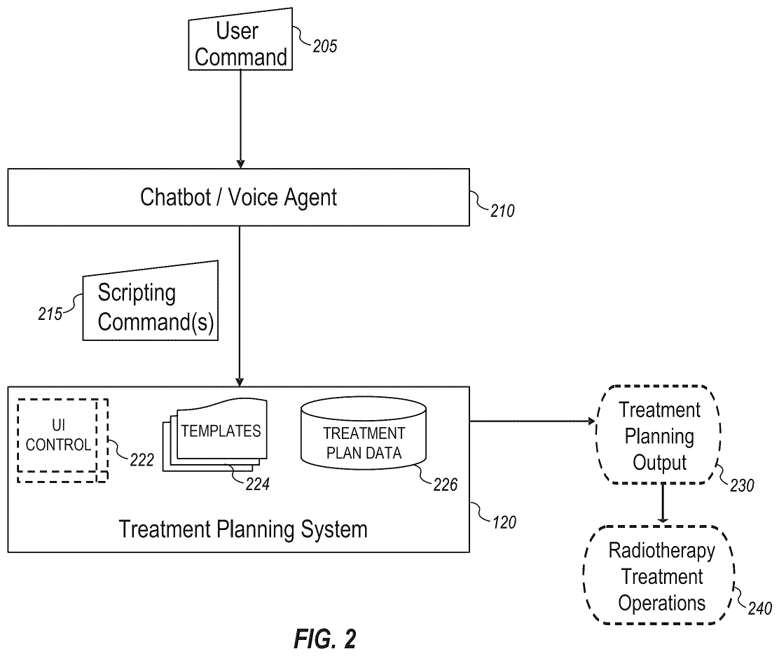

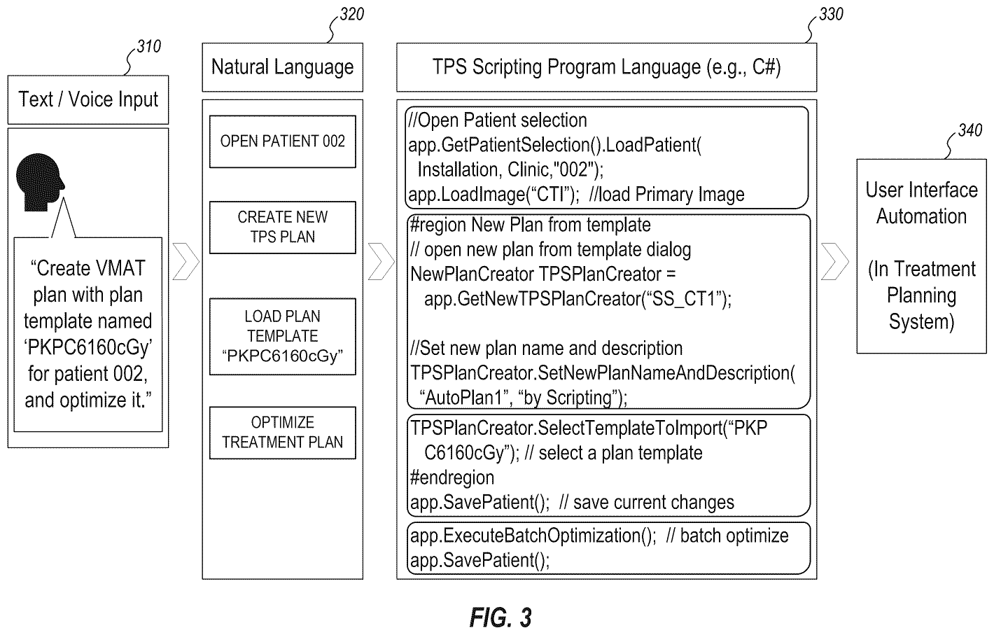

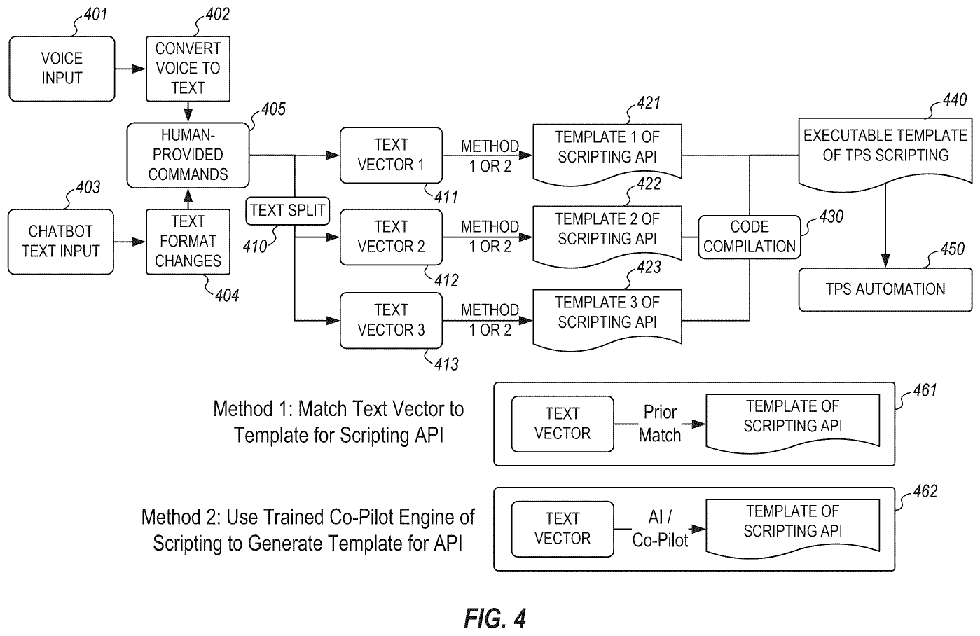

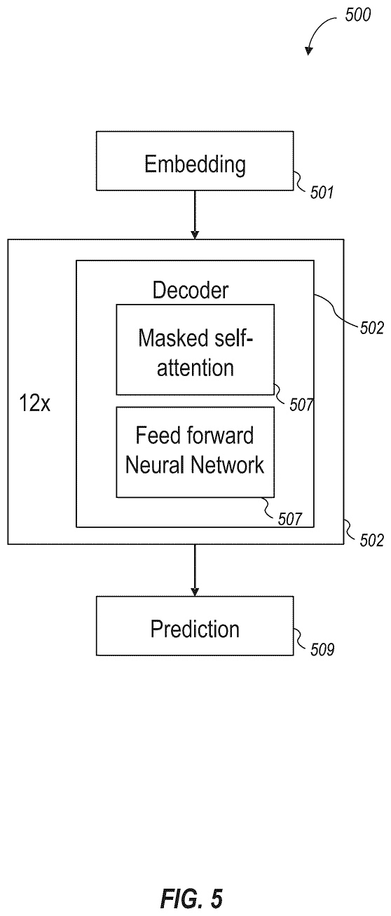

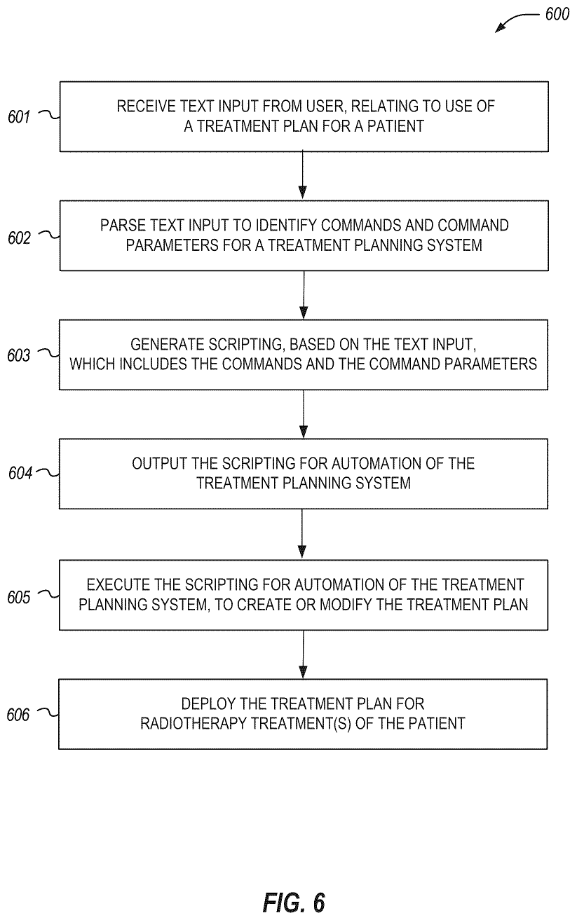

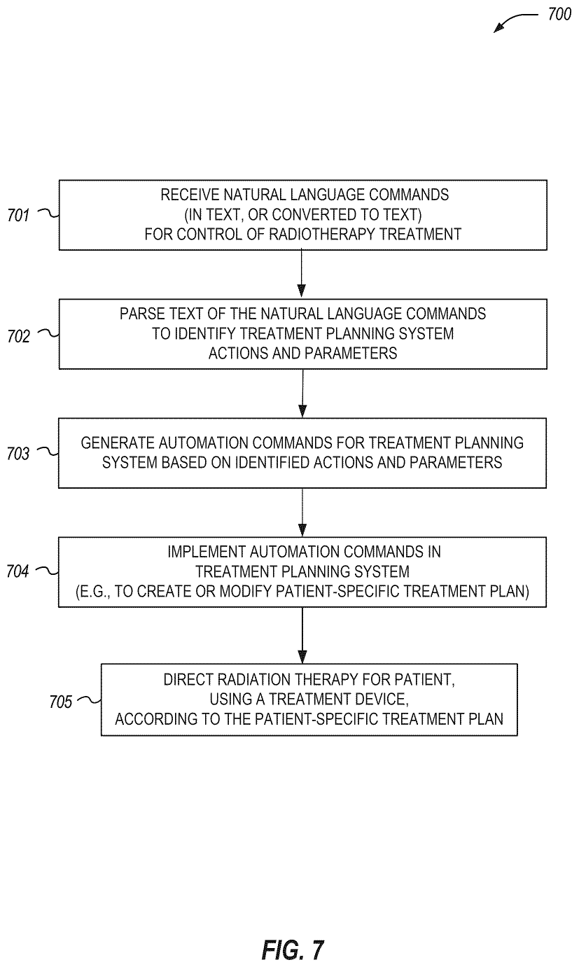

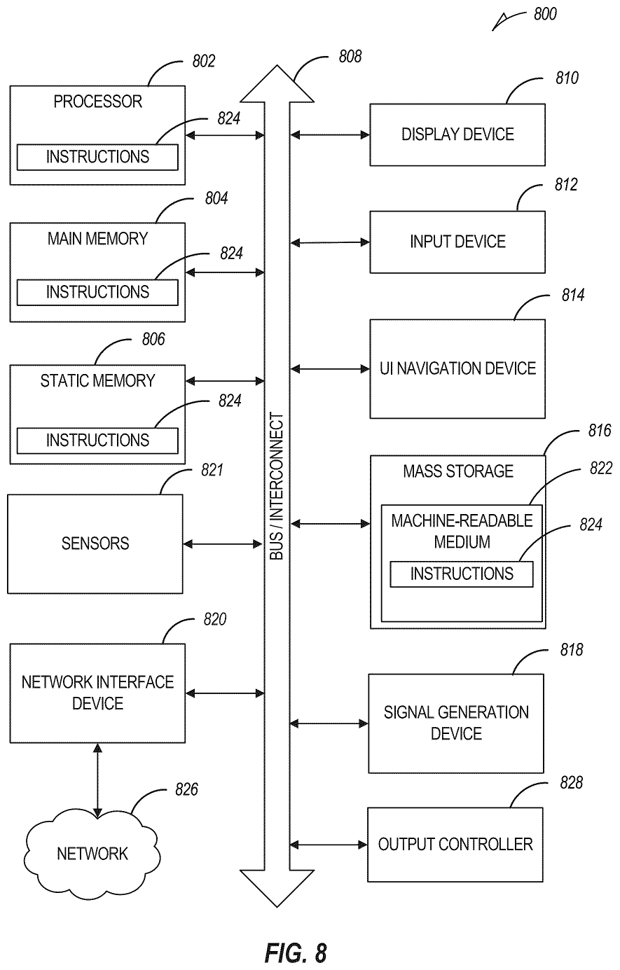

Other details of an implementing computing system are provided in FIG. 8. More details of the treatment planning automation workflow 130 are provided below with reference to FIGS. 2 and 3. Likewise, more details of the natural language processing 132, chatbot functionality 134, and scripting functionality 136 used in the treatment planning automation workflow 130 are provided below with reference to FIGS. 4 and 5. FIGS. 6 and 7 additionally detail how a treatment plan may be further evaluated, selected, optimized, and deployed in a radiotherapy planning system and then to a radiotherapy machine, as a result of the treatment planning automation workflow 130.

Radiotherapy treatment planning may arise in a variety of contexts. Different users or clinical centers may use different protocols or recipes to arrive at suitable treatment plans, and the outcomes of the devised treatment plans may be dependent on the experience of the healthcare professional. Automation in treatment planning systems has generally improved the efficiency of users (dosimetrists) while preserving the treatment quality. However, even with partially automated systems, the outcomes and optimizations of particular radiotherapy plans are often based on the skill of the user to implement scripting and programming.

The improvements of a treatment planning system described herein can be distinguished from prior automation solutions involving scripting. Automation of treatment planning systems via scripting has standardized processes and enabled staff to become proficient in more complex techniques, such as in connection with the increased utilization of Volumetric Modulated Arc Therapy (VMAT) procedures and related VMAT planning software. However, such scripting often requires the use of programming languages such as C sharp (C#) or Python, or the knowledge of how to invoke specific programming libraries and functions with the use of visual scripting.

Most clinicians such as a physicist, dosimetrist, or therapist have no background in computer science, so the learning curve is very steep to effectively invoke scripting. In addition, the use of programming languages in scripts adds an enhanced hurdle for debugging and commissioning the scripts. These two drawbacks have presented obstacles to the widespread adoption of scripting and automation procedures. The following techniques introduce approaches for the conversion of natural language commands into scripting and automation to address these drawbacks and related technical problems in automation.

FIG. 2 depicts a use case for invoking the treatment planning automation workflow 130. First, a user command 205 is received by a text chatbot or voice agent 210. The text chatbot or voice agent 210 applies logic to convert the user command 205 into one or more scripting commands 215. Based on the type of user command and the type of action to be invoked, each user command may result in the creation of a single scripting command or multiple scripting commands. Additionally, in some settings, multiple user commands (or a series of user commands) may cause the creation or modification of multiple scripting commands.

The one or more scripting commands 215 may affect a variety of aspects of operation of the treatment planning system 120. These include but are not limited to, automation of user interface control 222, use of radiotherapy planning templates 224, and the access, modification, creation, or update of treatment plan data 226. In the following examples, the treatment planning system may automate the creation or modification of a treatment plan for a particular patient in the treatment plan data 226, based on a set of one or more scripting commands 215 that invokes the user interface control 222 and one or more templates 224. The treatment planning system 120 may also provide application programming interfaces (APIs) and other programmatic features (not shown) that can be invoked and automated from the one or more scripting commands 215.

The automation of the treatment planning system 120 is thus used to produce one or more treatment plans, as treatment planning output 230 (e.g., in the form of data for the one or more treatment plans and related treatment control data). The treatment planning output 230 then can be used to control the radiotherapy treatment operations 240.

The presently disclosed approaches for scripting and automation provide benefits over existing uses of automated treatment planning tools for radiotherapy. Such planning automation often relies on some form of intelligence to translate clinical needs into treatment plans, such as by directly suggesting or inferring some treatment output. Some examples include the use of knowledge-based planning (KBP), and multicriterial optimization (MCO). For instance, KBP involves use of a library of clinically accepted, high-quality plans. KBP can be used to suggest how good a plan could be by comparing the new patient's anatomy with the plan library, allowing a planner to learn from experienced colleagues' suggestions. On the other hand, some approaches of MCO include sequentially trying to protect an organ at risk (OAR) from treatment, as best as possible, without compromising the target coverage by substituting the planner in the typical trial-and-error procedure. MCO typically looks for optimal solutions belonging to the so-called Pareto surface, meaning that a plan cannot be further improved on any objective without degrading the results on at least one of the others. This surface navigation may be performed by some treatment planning systems with an a priori MCO, often proposing only one planning solution respecting the listed requests. In a posteriori approach, the user can navigate between the generated multiple plans to choose the plan that best meets the clinical requests.

The present approaches for translation of human-language user commands into treatment planning system commands can provide an enhanced approach not achievable by the sole use of KBP and MCO automations. However, the scripting and automation approaches discussed herein may be used in combination with approaches of KBP and MCO automation. For instance, user commands may be used to direct the usage and outcomes from KBP or MCO, or to apply aspects of validation or optimization in some automated planning outcome assisted by KBP or MCO.

FIG. 3 shows a sequence of operations for treatment automation (e.g., with a use case involving the treatment planning automation workflow 130, demonstrating the creation of scripting commands 215). This sequence of operations includes an example of an automation workflow for chatbot/voice control-driven automation in a treatment planning system. For example, the treatment planning system 120 may be adapted to produce an automated VMAT plan and optimize this VMAT plan, based on the use of some template for a patient.

First, text or voice input 310 is collected, which includes a user command relating to the radiotherapy planning operations. The user command may be provided by a user in the form of a speech recording or audio capture, or in the form of a text string. When the command is in the form of a speech recording or audio capture, the speech can be transcribed into a text string using an automatic speech recognition (ASR) tool (e.g., implemented in a voice-to-text or speech-to-text engine). This allows a user to speak/type a command such as, “Please help me create a VMAT plan with plan template named ‘PKUNPC6160cGy’ for patient 002, then calculate and optimize it”.

Next, the text or voice input is provided to natural language processing of a chatbot or other language engine. In an example, the natural language processing 320 may be implemented by an AI language model chatbot (e.g., a generative Large Language Model (LLM) neural network) that is specially trained to translate clinical needs into inputs for software from natural language interactions. For instance, an algorithm of the chatbot can receive and parse the text to intelligently identify different commands, to identify and generate different actions that correspond to different scripting commands of a workflow (e.g., scripting commands that must be used in a valid order, piece by piece).

As shown, radiotherapy plan templates and parameters for the radiotherapy plan templates can be automatically selected and generated by the chatbot, including with the use of API or system calls that are combined together into a script. A code compilation is then created at 330, which in this example is configured to produce scripting in a programming language understandable by or usable with the treatment planning system (here, C#scripting language to be executed by a code interpreter of the treatment planning system). The depicted scripting provides user interface automation 340 in the treatment planning system, and is shown as invoking actions in the user interface to select a patient, load an image, instantiate a new plan, establish properties of a new plan, select a plan template for the plan, optimize the plan based on the plan template, and save the plan, among other actions.

This script and other scripting commands can be automatically executed, to implement automation in the user interface or APIs of the treatment planning system 120. For instance, a particular script may cause the treatment planning system 120 to customize a radiotherapy plan for a specific patient (e.g., a VMAT plan), and invoke associated processing actions to optimize the generated plan. The automation 350 may include other interpreting of the commands in connection with the user interface automation 340 in the treatment planning system.

FIG. 4 depicts a detailed processing workflow of how the treatment planning automation workflow 130 can convert human commands, provided in voice input 401 or text input 403, into executable commands. In particular, the depicted processing workflow demonstrates how text values that are input from a human are split into specific vectors, and used to invoke specific scripting commands.

As shown, the voice input 401 or the text input 403 is received via a chatbot or provided to a chatbot. The voice input 401 may be converted into text with a voice-to-text function 402, and the text input 403 may be converted or modified based on text formatting function 404. The output of either function 402 or 404 produces human-provided commands 405 in text format for further processing.

The human-provided commands 405 are then provided to a text split function 410, to divide portions of the text into individual text vectors 411, 412, 413. In an example, the text split function 410 is achieved via Python coding. A text vector, also known as an embedding, is a representation of some portion of the text, often with some numerical value. Vectors are used to capture the semantic meaning of the text, such that words and phrases with similar meanings will have vectors that are close to each other in a high-dimensional space.

Each of these text vectors 411, 412, 413 may be used to produce different scripting actions or commands. For instance, a first vector may correspond to one or more command with a first API, a second vector may correspond to one or more command with a second API, and so forth. The text vectors 411, 412, 413 as shown are provided into respective scripting API templates (template 1 421, template 2 422, template 3 433) used to generate different code functions. In some examples, an individual vector may be used to invoke multiple APIs or provide multiple parameters to an individual API. Other forms of natural language processing may also be used.

One or multiple methods may be used to convert the respective text vectors into code based on code templates that are designed for invoking specific functions or actions with a scripting API. As a first example of text processing 461, a text vector can be translated to the use of a corresponding scripting API, based on similarity metrics that evaluate which API is the most relevant to the function. This can be performed with a fuzzy match approach, where API parameters can be recognized from the text vectors and converted into scripting commands.

As a second example of text processing 462, a text vector can be translated to the use of a corresponding scripting API, based on generation or prediction from an AI engine or co-pilot engine (a specialized engine that suggests code completion). Here, each text vector can be provided as an input to the engine, where the engine model provides code to invoke the scripting API as output. This co-pilot model may be similar to other types of programming engines that generate code based on text command inputs.

The code results from all of the text vectors can be combined or merged. As shown, a code compilation function 430 may be invoked to combine the results of the templates 421, 422, 423. This produces an executable template of scripting commands 440, in a programming language format. These commands can be executed to directly control one or more workflow of the treatment planning system 120 with resulting automation 450.

The automation system and methods discussed above provide a number of technical advantages. One significant advantage is that the generation of scripting from generative chatbots can tremendously reduce the complexity for users who do not have any programming background to achieve the automation workflow in a treatment planning system. Even for skilled users with a programming background, the generation of scripting from generative chatbots can provide an advanced capability to reduce time for deploying and debugging scripts. Further, both skilled and unskilled users can use natural language to directly control a treatment planning system and achieve automation of a variety of functions and capabilities. This may also assist users who are able to easily articulate, in natural language, what types of modifications or changes need to be made to a patient radiotherapy treatment plan (including real-time or on-the-fly changes) and related therapy constraints.

In further examples, users can provide human commands to not only create treatment plans, but also invoke a variety of other treatment plan actions. Such commands could be invoked with real-world scenarios such as sending a message to the system to ask it to run an automation, such as, “create a treatment plan”, “export plan DICOM data to {a proprietary format}”, “print out the treatment plan report”, etc. Such actions may be automated without the need for training models that are customized to specific patients, facilities, or specific types of treatment plans or outcomes. This enables the customization of treatment plans (including those hosted or maintained by a medical organization) by other users who are skilled or unskilled. This use of generative AI can be used to translate needs into a format that the software will understand to produce the best outputs.

Although the examples above were discussed with reference to treatment planning software, other types of automation and control of a client application may be provided. A client application in this context can refer to additional types of a computer program configured to process medical data via one or more features (e.g., functions, methods, services, or components provided or controlled by the client application). Thus, a client application can include any software that can access and analyze medical data in connection with radiotherapy, such as any one or more of patient data, medical imaging data, treatment plan information, dose-volume histograms (DVHs), dose information, dosimetric metrics, and clinical outcomes, and the like.

FIG. 5 shows a schematic illustration of an example language model 500 usable by a chatbot. A language model is configured to predict and/or generate plausible words for a given input. In an example, the language model 500 comprises a neural network, and the language model 500 is based on a transformer network 502. A transformer network is a type of neural network that uses a self-attention mechanism. The transformer model is described in Vaswani et al. “Attention is all you need.” Advances in neural information processing systems 30 (2017), for example.

In an example, the language model 500 comprises a Generative Pre-trained Transformer (GPT) network (configured as the transformer network 502). Language models comprising a GPT network may be referred to as Large Language Model (LLM). Transformer models are able to handle long term dependencies in text and/or natural language processing and may be used in chatbots. The chatbot may comprise other components (not shown) such as components for preprocessing text before it is fed to the language model, or components for postprocessing the output of the language model before returning a response to the user. Preprocessing operations include operations such as tokenization (i.e., the splitting of a text string into smaller sequences of characters, referred to as tokens). The postprocessing operations depend on whether the language model is used for natural language inference, question-answering tasks, similarity assessment tasks, classification tasks and so on.

The text and position embedding 501 represents the input to the Transformer network 502 (or a similar GPT network). The text and position embedding 501 corresponds to an encoded form of the user query described herein. To obtain the text and position embedding, bytepair encoding (BPE) may be used to covert the text string corresponding to the user query into a sequence of tokens. The tokens are selected from a predetermined vocabulary of tokens. The size of the vocabulary is denoted by V. For example, the vocabulary comprises between 32,000 and 64,000 tokens (i.e., V may be between 32,000 and 64,000). Each token is represented by a token embedding. The dimensionality, D, of the token embedding may be any of 768, 1024, 1280 or 1600, for example. The vocabulary may be represented by a token embedding matrix of size V×D. The token embedding matrix may be denoted Wₑ. Further, for each token in the sequence, a positional encoding vector (which indicates the order of the tokens in the sequence of tokens provided to the transformer), having the same dimension as the token embedding (D), is added to the token embedding. For example, the positional encoding vector may represent any one of 1024 positions in the input sequence. The positional encoding vector may be a learned (i.e., it comprises parameters that are determined during training). The positional encoding vector is denoted Wp. The length of the input sequence (e.g., 1024) may be referred to as the context size. The text and position embedding 501 is obtained based on the token embedding matrix and the positional encoding vector. The text and position embedding 501 comprises a sequence of vectors, representing the user query, where each vector has length D.

The language model 500 shown in FIG. 5 comprises a transformer network 502. The transformer network 502 comprises a stack of twelve (12×) decoder blocks 503. Each decoder block 503 comprises a masked self-attention layer 505 and a feed forward neural network 507. The first decoder block receives a sequence of vectors, each vector representing a token, from the text and position embedding 501. Layer normalization (not shown) may be provided at the input of each masked self-attention layer 505 and at the input of each feed forward neural network 507. After the final (twelfth, in this case) self-attention layer, a further layer normalization may be included. The purpose of layer normalization is to provide smoother gradients, faster training and improved accuracy.

For ease of explanation, consider a sequence of four tokens, represented by four vectors x₁, x₂, x₃ and x₄. Each of x₁, x₂, x₃ and x₄ has a length D. The tokens are fed into the masked self-attention layer 505 of the first decoder block 503. For each token, a query vector, q, a key vector, k, and a value vector, v, is obtained. The query, key, and value vectors for each token are obtained by multiplying weight matrices W^{Q}, W^{K} and W^{V} by the token vector. The weight matrices W^{Q}, W^{K} and W^{V} have a size of D. The weight matrices comprise weights that are determined during the training of the network. For each token i, the query vector q; is multiplied (dot product) by the key vectors (for all tokens) to get a score that indicates how well the other tokens match with the current token. In masked self-attention, the scores for future tokens (future tokens are those tokens that appear after the current token in the sequence) are set to 0. The value vector, vi, for each token are multiplied by their respective scores and then summed up to obtain a vector denoted as z. For x₁, x₂, x₃ and x₄, corresponding vectors z₁, z₂, z₃, and z₄ are obtained. z₁, z₂, z₃, and z₄ are the outputs of the masked self-attention layer 505. These are then presented to the feed forward neural network 507 layer of the decoder block. The feed forward NN is a fully-connected NN where the vector z₁ for each token i is projected (by multiplying by further matrices—those matrices comprise weights that are determined during the training of the network) onto a result vector Rⱼ.

The result vectors for each decoder block 503 are passed on as an input to the next decoder block. Each block 503 comprises its own weight matrices (determined during model training). For the final decoder block, the result vectors are used to derive the prediction 509 of the transformer model. In more detail, each result vector is multiplied by the token embedding matrix. The result of this multiplication corresponds to a score for each of the V tokens in the vocabulary. This result may be taken as the prediction 509. In some examples, the token with the highest score is selected and used to form the prediction. In another example, the top scoring k tokens are considered. For example, k=40. The model iterates through all tokens until the end of the sequence is reached.

The language model 500 may be trained as follows. The training procedure may comprise two stages: (i) unsupervised pre-training, and (ii) supervised fine-tuning. In the unsupervised pre-training stage, given an unsupervised corpus of tokens [symbol] ={u₁, . . . uₙ}, a standard language modelling objective is used to maximize a likelihood function. For example the likelihood function is:

$${L}_{1} ( 𝒰 ) = Σ log P ( {u}_{i} \vert {u}_{i - k} , … , {u}_{i - 1} : ⊖ ) \qquad EQUATION 1$$

Where k is the context size, and the conditional probability P is modelled using a neural network with parameters Θ. The corpus of tokens may be obtained from a dataset comprising ˜8 million documents (that corresponds to 40 GB of text). An example is the WebText corpus by OpenAI. Alternatively, the corpus of tokens may be obtained from a dataset such as the BooksCorpus. The parameters may be determined using stochastic gradient descent. For example, an Adam optimization scheme (with a maximum learning rate of 2.5e-4) may be used.

The language model 500 applies the following operation to the inputted token to produce an output distribution over target tokens:

$${R}_{o} = {UW}_{e} + {W}_{p} \qquad EQUATION 2$$

$${R}_{l} = transformer ( {R}_{l - 1} ) ∀ / ∈ [ 1 , n ]$$

$$P ( u ) = softmax ( {R}_{n} {W}_{e} )$$

Here, U is the vector of tokens, n represents the number of decoder blocks (n=12 in FIG. 5), R₀ represents the embedding 501 that is fed into the transformer network 502, R_{I}=transformer (R_{I-1}) represents the operation applied by the I^{th} decoder block of the transformer to the result vector R_{I-1} from the (I−1)^{th} block, and P(u)=softmax(RₙWₑ) represents the application of the softmax function to the product of the result vector Rₙ from the last decoder block and the token embedding matrix Wₑ to provide an output distribution.

In the supervised fine-tuning stage, the pre-trained model from the first stage is further trained using a labelled dataset [symbol] . The dataset [symbol] comprises a sequence of input tokens c¹, . . . , c^{m} and a label y. In an example, the label y is the ground truth of the sequence of input tokens, and may include the known command and known command parameter of the treatment plan system corresponding to the sequence of input tokens. The label y can be obtained by manually labelling by human and/or with machine assisted cognition. The inputs are fed into the pre-trained model to obtain a result vector R^{m}ₙ from the last block of the transformer. The result vector is R^{m}ₙ is fed into a further linear output layer with parameters W_{y} to obtain a prediction for y as follows: P(y|c¹, . . . , c^{m})=softmax(R^{m}ₙW_{y}).

The objective to maximize in the supervised fine-tuning stage is:

$${L}_{2} ( 𝒞 ) = Σ log P ( y \vert {c}^{1} , … , {c}^{m} ) \qquad EQUATION 3$$

As for the first stage, the parameters may be determined using stochastic gradient descent. For example, an Adam optimization scheme (with a maximum learning rate of 2.5e-4) may be used.

The model described in FIG. 5 comprises a single attention head. Alternatively, the model may comprise multiple attention heads.

In alternative examples, the language model 500 comprises an architecture as described in Radford, A, et al. “Improving Language Understanding by Generative Pre-Training.” OpenAI.

In alternative examples, the language model 500 comprises a GPT-2 architecture as described in Radford et al. “Language models are unsupervised multitask learners.” OpenAI blog 1.8 (2019): 9.

In alternative examples, the language model 500 comprises a GPT-3 architecture as described in Brown, T. et al. “Language models are few-shot learners”. Advances in neural information processing systems, 33, 1877-1901 (2020).

Yet in another alternative example, the language model 500 comprises a transformer-based architecture as described in Touvron et al. “Llama 2: Open foundation and fine-tuned chat models.” arXiv preprint arXiv:2307.09288 (2023).

Yet in another alternative example, the language model 500 comprises architectures based on GPT-4 or PaLM2.

Yet in another alternative example, the language model 500 comprises recurrent neural network (RNN) or a long short-term memory (LSTM) network.

FIG. 6 is a flowchart 600 of an example method for generating automation commands for a treatment planning system used with radiotherapy treatment. This method may include variations based on the text processing and generation examples discussed above with reference to FIGS. 2-4.

At 601: Receive text input from user, with this text input including information relating to use of a treatment plan for a patient. In some examples, this text input from the user is derived from audio, and the method further comprises: obtaining the audio from the user; and converting the audio into the text input with a voice-to-text engine.

In some examples, the receiving of the text input includes use of at least one chat session with the generative chatbot, such that the at least one chat session includes multiple questions and responses provided between the generative chatbot and the user relating to the radiotherapy treatment of the patient. For instance, the generative chatbot may include and use a trained language model, such as a generative pre-trained transformer (GPT) network.

At 602: Parse text input to identify commands and command parameters for a treatment planning system. In one example, the parsing of the text input includes splitting the text input into vectors, and identifying at least one command of the scripting based on a relevance match of the vectors. In another example, the parsing of the text input includes splitting the text input into vectors, parsing the vectors with an artificial intelligence (AI) coding engine, and identifying at least one command of the scripting with a coding engine.

In some examples, where a generative chatbot is used to obtain text, the method may include receiving medical data associated with the patient, and determining the commands and the command parameters by the generative chatbot based on the medical data associated with the patient. Such medical data may include at least one of: electronic medical records, electronic health records, patient data, treatment plans, treatment plan templates, and medical imaging data.

At 603: Generate scripting (e.g., with a generative chatbot), based on the text input, which includes the commands and the command parameters. In an example, the commands of the treatment planning system are provided for programmatic use of multiple application programming interfaces (APIs) of the treatment planning system, and the command parameters are provided as inputs for the programmatic use of the multiple APIs. In an example, the scripting is generated with a generative chatbot, and this generative chatbot operates based on a trained language model. For instance, the trained language model may be trained based on known commands and known command parameters of the treatment planning system associated with creating and modifying radiotherapy treatment plans. The commands and command parameters may correspond to known (e.g., previously used) values that invoke specific commands of the multiple APIs from scripting, based on prior treatment planning use cases and patient data examples. The trained language model may be further trained based on multiple restrictions and instructions associated with creating and modifying of the radiotherapy treatment plans, including specific actions that are required or prohibited for generating a radiotherapy treatment plan in the prior treatment planning use cases and the patient data examples. In still further examples, the trained language model is further trained to invoke one or more cost functions for establishing the radiotherapy treatment plan for the radiotherapy treatment of the patient, based on the multiple restrictions and instructions.

At 604: Output the scripting for automation of the treatment planning system, in connection with establishing the radiotherapy treatment plan for the radiotherapy treatment of the patient. The commands provided by the scripting may include use of a treatment plan template, including where the use of the treatment plan template includes customization of the radiotherapy treatment plan based on the treatment plan template, and optimization of the radiotherapy treatment plan.

In some examples, the outputting of the scripting includes presenting the scripting, and receiving a modification to the scripting from the user. The scripting may include source code provided in a programming language format, and outputting the scripting may include providing an output of the source code for automation of a graphical user interface. In other examples, the scripting may include output as a visual script, and outputting the scripting may include providing an output of the visual script in a graphical user interface.

At 605: Execute the scripting for automation of the treatment planning system, to create or modify the treatment plan. The execution of the scripting may result in the creation, modification, and optimization of the treatment plan, as discussed above.

At 606: Deploy the treatment plan for radiotherapy treatment(s) of the patient, including with the control of a respective radiotherapy machine based on the treatment plan. The deploying of the treatment plan may occur in connection with the aspects discussed with reference to FIG. 1, above.

FIG. 7 is a flowchart 700 of an example method for implementing automation in a radiotherapy treatment planning system. This method may be coordinated in combination with the operations of flowchart 600, and may include variations based on the text processing and generation examples discussed above with reference to FIGS. 2-4.

At 701: Receive natural language commands in text from a user, with the natural language commands including information relating to an intended or planned use of a radiotherapy treatment for a patient. In some examples, the natural language commands are received from the user in an audio format, and the method includes converting audio of the natural language commands into the text.

At 702: Parse text of the natural language commands to identify treatment planning system actions and parameters. In an example, the parsing of the text includes splitting the text into vectors, and identifying the automation commands based on a relevance match of the vectors. In an example, the parsing of the text includes splitting the text into vectors, parsing the vectors with an artificial intelligence (AI) coding engine, and identifying the automation commands with the coding engine.

At 703: Generate automation commands for treatment planning system based on identified actions and parameters. In some examples, prior to generating the automation commands, the method may determine that the natural language commands are not directly executable to control the treatment planning system, and thus must be converted into another format as automation commands. The automation commands may be provided in scripting, and the automation commands may be provided as source code within the scripting in a programming language format. The automation commands may invoke multiple application programming interfaces (APIs) of the treatment planning system within the scripting, with the parameters being provided with programmatic use of the multiple APIs. In some examples, the automation commands include use of a radiotherapy plan template, and wherein the use of the radiotherapy plan template includes customization of a radiotherapy treatment plan for the patient based on the radiotherapy plan template, and optimization of the radiotherapy treatment plan. Also in some examples, the automation commands invoke at least one command to optimize a radiotherapy treatment plan in the treatment planning system, based on the natural language commands.

The generation of the automation commands may be performed using a generative chatbot (e.g., a chatbot that includes a language model provided from a generative pre-trained transformer (GPT) network). The natural language commands also may be received from the user using a chat session with this generative chatbot, such as with a chat session includes multiple questions and responses provided between the generative chatbot and the user. In further examples, the generative chatbot may operate with a trained language model that is trained based on multiple restrictions and instructions relating to generation of a radiotherapy treatment plan. Additionally, the trained language model may be trained to invoke one or more cost functions for optimization of the radiotherapy treatment plan based on the multiple restrictions and instructions. Additionally, in some examples, the method may include receiving medical data associated with the patient, and determining the automation commands are determined by the generative chatbot based on the medical data associated with the patient. Such medical data may include at least one of: electronic medical records, electronic health records, patient data, treatment plans, treatment plan templates, and medical imaging data.

At 704: Implement automation commands in treatment planning system (e.g., to create or modify patient-specific treatment plan). In scenarios where scripting is generated, implementing the automation commands to programmatically control the treatment planning system includes executing the scripting. In an example, the results of implementing the automation commands may include outputting a radiotherapy treatment plan for the radiotherapy treatment, with the automation commands to programmatically control the treatment planning system being used to create the radiotherapy treatment plan, based on the natural language commands. In another example, modifying the radiotherapy treatment plan associated with the patient, involves using the automation commands to programmatically control the treatment planning system to update the radiotherapy treatment plan, based on the natural language commands.

At 705: Direct radiation therapy for patient, using a treatment device, according to the patient-specific treatment plan. The deploying of the treatment plan may occur in connection with the aspects discussed with reference to FIG. 1, above.

FIG. 8 is a block diagram of an example of an apparatus, device, system or machine 800 upon which any one or more of the techniques (e.g., methods) discussed herein may perform. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act or operate as a peer machine in peer-to-peer (P2P) (or other distributed) network environment. The machine 800 may be a personal computer (PC), a tablet PC, server computer, a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 800 may receive a user command by way of any of input device 812, UI navigation device 814, display device 810 or microphone (optional) as described herein. The machine 800 may output an indication that an action has been performed by way of any of display device 810 and signal generation device 818.

The machine 800 may output processed medical data in machine readable medium 822 as described herein. The machine 800 may be used to implement the operations performed by the client application and/or the chatbot, and related data processing systems involved in translating natural language commands into automation commands.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out or conduct a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out or conduct portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, field programmable gate array (FPGA), or any combination thereof), a main memory 804 and a static memory 806, some or all of which may communicate with each other via an interlink (e.g., bus) 830. The machine 800 may further include a display device 810, an input device 812 (e.g., a keyboard or other alphanumeric input device), and a user interface (UI) navigation device 814 (e.g., a mouse). In an example, the display device 810, input device 812 and UI navigation device 814 may be a touch screen display. Optionally, the machine 800 includes a microphone (e.g., to receive audio of verbal instructions that are converted to text input). The machine 800 may additionally include a storage device 808 (e.g., drive unit or other similar mass storage device or unit), a signal generation device 818 (e.g., a speaker), a network interface device 820 connected to a network 826, and one or more sensors 821, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 808 may include a machine readable medium 822 on which is stored one or more sets of data structures or instructions 824 (e.g., software) embodying or used by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 808 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824. The term “machine readable medium” may include any medium that is capable of storing, encoding, or carrying instructions for execution by the machine 800 and that cause the machine 800 to perform any one or more of the techniques of the present disclosure, or that is capable of storing, encoding, or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

Unless specifically stated otherwise, it is appreciated that throughout the description, discussions utilizing terms such as “receiving”, “determining”, “comparing”, “enabling”, “maintaining,” “identifying,”, “obtaining”, “accessing” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Additional aspects and features of the present disclosure are set forth in the following numbered examples:

Example 1 is a method for generating automation commands for a treatment planning system used with radiotherapy treatment, the method comprising: receiving text input from a user, the text input including information relating to a radiotherapy treatment plan of a patient; determining, based on parsing the text input, commands of the treatment planning system and command parameters to be used by the treatment planning system; generating scripting with a generative chatbot based on the text input, wherein the scripting includes the commands and the command parameters; and outputting the scripting for automation of the treatment planning system in connection with establishing the radiotherapy treatment plan for the radiotherapy treatment of the patient.

In Example 2, the subject matter of Example 1 optionally includes subject matter where the method further includes executing the scripting.

In Example 3, the subject matter of Example 2 optionally includes subject matter where the commands of the treatment planning system are provided for programmatic use of multiple application programming interfaces (APIs) of the treatment planning system, and wherein the command parameters are provided as inputs for the programmatic use of the multiple APIs.

In Example 4, the subject matter of any one or more of Examples 1-3 optionally include subject matter where the text input from the user is derived from audio, and wherein the method further comprises: obtaining the audio from the user; and converting the audio into the text input with a voice-to-text engine.

In Example 5, the subject matter of any one or more of Examples 1-4 optionally include subject matter where the generative chatbot operates based on a trained language model, and wherein the trained language model is trained based on known commands and known command parameters of the treatment planning system associated with creating and modifying radiotherapy treatment plans.

In Example 6, the subject matter of Example 5 optionally includes subject matter where the trained language model is further trained based on multiple restrictions and instructions associated with creating and modifying of the radiotherapy treatment plans.

In Example 7, the subject matter of Example 6 optionally includes subject matter where the trained language model is further trained to invoke one or more cost functions for establishing the radiotherapy treatment plan for the radiotherapy treatment of the patient, based on the multiple restrictions and instructions.

In Example 8, the subject matter of any one or more of Examples 1-7 optionally include subject matter where the commands include use of a treatment plan template, and wherein the use of the treatment plan template includes customization of the radiotherapy treatment plan based on the treatment plan template, and optimization of the radiotherapy treatment plan.

In Example 9, the subject matter of any one or more of Examples 1-8 optionally include subject matter where parsing the text input includes splitting the text input into vectors, and identifying at least one command of the scripting based on a relevance match of the vectors.

In Example 10, the subject matter of any one or more of Examples 1-9 optionally include subject matter where parsing the text input includes splitting the text input into vectors, parsing the vectors with an artificial intelligence (AI) coding engine, and identifying at least one command of the scripting with the coding engine.

In Example 11, the subject matter of any one or more of Examples 1-10 optionally include subject matter where the scripting includes source code provided in a programming language format, and wherein outputting the scripting includes providing an output of the source code for automation of a graphical user interface.

In Example 12, the subject matter of any one or more of Examples 1-11 optionally include subject matter where the scripting includes a visual script, and wherein outputting the scripting includes providing an output of the visual script in a graphical user interface.

In Example 13, the subject matter of any one or more of Examples 1-12 optionally include subject matter where outputting the scripting includes: presenting the scripting; and receiving a modification to the scripting from the user.

In Example 14, the subject matter of any one or more of Examples 1-13 optionally include subject matter where receiving the text input includes use of at least one chat session with the generative chatbot, and wherein the at least one chat session includes multiple questions and responses provided between the generative chatbot and the user relating to the radiotherapy treatment of the patient.

In Example 15, the subject matter of Example 14 optionally includes subject matter where the generative chatbot includes a language model.

In Example 16, the subject matter of Example 15 optionally includes subject matter where the language model includes a generative pre-trained transformer (GPT) network.

In Example 17, the subject matter of any one or more of Examples 1-16 optionally include subject matter where the method further comprises: receiving medical data associated with the patient; wherein the commands and the command parameters are determined by the generative chatbot based on the medical data associated with the patient.

In Example 18, the subject matter of Example 17 optionally includes subject matter where the medical data includes at least one of: electronic medical records, electronic health records, patient data, treatment plans, treatment plan templates, and medical imaging data.

Example 19 is a system for generating automation commands for a radiotherapy treatment planning system, the system comprising: processing circuitry; and memory, including instructions stored thereon, which, when executed by the processing circuitry, cause the processing circuitry to perform any of the methods of Examples 1 to 18.

Example 20 is a non-transitory computer-readable medium with instructions stored thereon that, when executed by a processor of a computing device, cause the processor to perform the methods of any of Examples 1 to 18.

Example 21 is a method for implementing automation in a treatment planning system, the method comprising: receiving natural language commands in text from a user, the natural language commands including information relating to intended use of a radiotherapy treatment for a patient; parsing the text of the natural language commands to identify actions to be performed in the treatment planning system and parameters to be used by the actions to be performed in the treatment planning system; generating automation commands, using a generative chatbot, the automation commands to control the treatment planning system based on the actions and the parameters; and implementing the automation commands to programmatically control the treatment planning system, in connection with computerized planning of the radiotherapy treatment for the patient.

In Example 22, the subject matter of Example 21 optionally includes outputting a radiotherapy treatment plan for the radiotherapy treatment, wherein the automation commands to programmatically control the treatment planning system are used to create the radiotherapy treatment plan, based on the natural language commands.

In Example 23, the subject matter of any one or more of Examples 21-22 optionally include modifying a radiotherapy treatment plan associated with the patient, wherein the automation commands to programmatically control the treatment planning system are used to update the radiotherapy treatment plan, based on the natural language commands.

In Example 24, the subject matter of any one or more of Examples 21-23 optionally include subject matter where the automation commands invoke at least one command to optimize a radiotherapy treatment plan in the treatment planning system, based on the natural language commands.

In Example 25, the subject matter of any one or more of Examples 21-24 optionally include subject matter where the automation commands are provided in scripting, and wherein implementing the automation commands to programmatically control the treatment planning system includes executing the scripting.

In Example 26, the subject matter of any one or more of Examples 21-25 optionally include subject matter where the automation commands are provided as source code in a programming language format.

In Example 27, the subject matter of any one or more of Examples 21-26 optionally include subject matter where the automation commands invoke multiple application programming interfaces (APIs) of the treatment planning system, and wherein the parameters are provided with programmatic use of the multiple APIs.

In Example 28, the subject matter of any one or more of Examples 21-27 optionally include subject matter where the automation commands include use of a radiotherapy plan template, and wherein the use of the radiotherapy plan template includes customization of a radiotherapy treatment plan for the patient based on the radiotherapy plan template, and optimization of the radiotherapy treatment plan.

In Example 29, the subject matter of any one or more of Examples 21-28 optionally include subject matter where the natural language commands are received from the user in an audio format, and wherein the method further comprises converting audio of the natural language commands into the text.

In Example 30, the subject matter of any one or more of Examples 21-29 optionally include subject matter where parsing the text includes splitting the text into vectors, and identifying the automation commands based on a relevance match of the vectors.

In Example 31, the subject matter of any one or more of Examples 21-30 optionally include subject matter where parsing the text includes splitting the text into vectors, parsing the vectors with an artificial intelligence (AI) coding engine, and identifying the automation commands with the coding engine.

In Example 32, the subject matter of any one or more of Examples 21-31 optionally include subject matter where the generative chatbot operates with a trained language model that is trained based on multiple restrictions and instructions relating to generation of a radiotherapy treatment plan.

In Example 33, the subject matter of Example 32 optionally includes subject matter where the trained language model is trained to invoke one or more cost functions for optimization of the radiotherapy treatment plan based on the multiple restrictions and instructions.

In Example 34, the subject matter of any one or more of Examples 21-33 optionally include subject matter where the natural language commands are received from the user using a chat session with the generative chatbot, and wherein the chat session includes multiple questions and responses provided between the generative chatbot and the user.

In Example 35, the subject matter of Example 34 optionally includes subject matter where the generative chatbot includes a language model provided from a generative pre-trained transformer (GPT) network.

In Example 36, the subject matter of any one or more of Examples 21-35 optionally include prior to generating the automation commands, determining that the natural language commands are not directly executable to control the treatment planning system.

In Example 37, the subject matter of any one or more of Examples 21-36 optionally include receiving medical data associated with the patient; wherein the automation commands are determined by the generative chatbot based on the medical data associated with the patient.

In Example 38, the subject matter of Example 37 optionally includes subject matter where the medical data includes at least one of: electronic medical records, electronic health records, patient data, treatment plans, treatment plan templates, and medical imaging data.

Example 39 is a system for implementing automation in a radiotherapy treatment planning system, the system comprising: processing circuitry; and memory, including instructions stored thereon, which, when executed by the processing circuitry, cause the processing circuitry to perform any of the methods of Examples 21 to 38.

Example 40 is a non-transitory computer-readable medium with instructions stored thereon that, when executed by a processor of a computing device, cause the processor to perform the methods of any of Examples 21 to 38.

According to another example, there is provided a computer program comprising instructions which, when the program is executed by a computer, cause the computer to carry out any of the above examples. A computer program and/or the code for performing such methods may be provided to a system (such as the system according to the second aspect) on one or more computer readable media or, more generally, a computer program product.

The examples described herein are computer-implemented methods. Since some methods in accordance with examples can be implemented by software, some examples encompass computer code provided to a general purpose computer on any suitable carrier medium. The carrier medium can comprise any storage medium such as a floppy disk, a CD ROM, a magnetic device or a programmable memory device, or any transient medium such as any signal e.g., an electrical, optical or microwave signal. The carrier medium may comprise a non-transitory computer readable storage medium.

The present disclosure also relates to a system for performing the operations herein. This system may be specially constructed for the required purposes, or it may comprise a general purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved and other advantageous results attained. Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the disclosure should be determined with reference to the appended claims, along with the full scope of equivalents to which such claims are entitled.

What is claimed is:

1. A method for generating automation commands for a treatment planning system used with radiotherapy treatment, the method comprising:
receiving text input from a user, the text input including information relating to a radiotherapy treatment plan of a patient;
determining, based on parsing the text input, commands of the treatment planning system and command parameters to be used by the treatment planning system;
generating scripting with a generative chatbot based on the text input, wherein the scripting includes the commands and the command parameters; and
outputting the scripting for automation of the treatment planning system in connection with establishing the radiotherapy treatment plan for the radiotherapy treatment of the patient.

2. The method of claim 1, wherein the method further comprises executing the scripting.

3. The method of claim 2, wherein the commands of the treatment planning system are provided for programmatic use of multiple application programming interfaces (APIs) of the treatment planning system, and wherein the command parameters are provided as inputs for the programmatic use of the multiple APIs.

4. The method of claim 1, wherein the text input from the user is derived from audio, and wherein the method further comprises:
obtaining the audio from the user; and
converting the audio into the text input with a voice-to-text engine.

5. The method of claim 1, wherein the generative chatbot operates based on a trained language model, and wherein the trained language model is trained based on known commands and known command parameters of the treatment planning system associated with creating and modifying radiotherapy treatment plans.

6. The method of claim 5, wherein the trained language model is further trained based on multiple restrictions and instructions associated with creating and modifying of the radiotherapy treatment plans.

7. The method of claim 6, wherein the trained language model is further trained to invoke one or more cost functions for establishing the radiotherapy treatment plan for the radiotherapy treatment of the patient, based on the multiple restrictions and instructions.

8. The method of claim 1, wherein the commands include use of a treatment plan template, and wherein the use of the treatment plan template includes customization of the radiotherapy treatment plan based on the treatment plan template, and optimization of the radiotherapy treatment plan.

9. The method of claim 1, wherein parsing the text input includes splitting the text input into vectors, and identifying at least one command of the scripting based on a relevance match of the vectors.

10. The method of claim 1, wherein parsing the text input includes splitting the text input into vectors, parsing the vectors with an artificial intelligence (AI) coding engine, and identifying at least one command of the scripting with the coding engine.

11. The method of claim 1, wherein the scripting includes source code provided in a programming language format, and wherein outputting the scripting includes providing an output of the source code for automation of a graphical user interface.

12. The method of claim 1, wherein the scripting includes a visual script, and wherein outputting the scripting includes providing an output of the visual script in a graphical user interface.

13. The method of claim 1, wherein outputting the scripting includes:
presenting the scripting; and
receiving a modification to the scripting from the user.

14. The method of claim 1, wherein receiving the text input includes use of at least one chat session with the generative chatbot, and wherein the at least one chat session includes multiple questions and responses provided between the generative chatbot and the user relating to the radiotherapy treatment of the patient.

15. The method of claim 14, wherein the generative chatbot includes a language model.

16. The method of claim 15, wherein the language model includes a generative pre-trained transformer (GPT) network.

17. The method of claim 1, wherein the method further comprises:
receiving medical data associated with the patient;
wherein the commands and the command parameters are determined by the generative chatbot based on the medical data associated with the patient.

18. The method of claim 17, wherein the medical data includes at least one of:
electronic medical records, electronic health records, patient data, treatment plans, treatment plan templates, and medical imaging data.

19. A system for generating automation commands for a radiotherapy treatment planning system, the system comprising:
processing circuitry; and
memory, including instructions stored thereon, which, when executed by the processing circuitry, cause the processing circuitry to:
receive text input from a user, the text input including information relating to a radiotherapy treatment plan of a patient;
determine, based on parsing the text input, commands of the treatment planning system and command parameters to be used by the treatment planning system;
generate scripting with a generative chatbot based on the text input, wherein the scripting includes the commands and the command parameters; and
output the scripting for automation of the treatment planning system in connection with establishing the radiotherapy treatment plan for the radiotherapy treatment of the patient.

20. A non-transitory computer-readable medium comprising instructions stored thereon that, when executed by a processor of a computing device, cause the processor to generate automation commands for a radiotherapy treatment planning system, including operations that:
receive text input from a user, the text input including information relating to a radiotherapy treatment plan of a patient;
determine, based on parsing the text input, commands of the treatment planning system and command parameters to be used by the treatment planning system;
generate scripting with a generative chatbot based on the text input, wherein the scripting includes the commands and the command parameters; and
output the scripting for automation of the treatment planning system in connection with establishing the radiotherapy treatment plan for the radiotherapy treatment of the patient.
